(19) United States
(12) Patent Application Publication
(10) Pub. No.: US 2023/0000683 A1
(43) Pub. Date: Jan. 5, 2023

(54) SKIN CARE APPARATUS

(71) Applicant: LG ELECTRONICS INC., Seoul (KR)

(72) Inventors: Munseong KANG, Seoul (KR); Gueisam LIM, Seoul (KR); Sangwon KIM, Seoul (KR); Heejung KIM, Seoul (KR); Jungwook KIM, Seoul (KR); Dongwon KIM, Seoul (KR)

(73) Assignee: LG ELECTRONICS INC., Seoul (KR)

(21) Appl. No.: 17/780,439

(22) PCT Filed: Jan. 16, 2020
(86) PCT No.: PCT/KR2020/000773

§ 371 Date: May 26, 2022

(30) Foreign Application Priority Data
Nov. 27, 2019 (KR) 10-2019-0153924

(51) Int. Cl. A61F 9/04; A61N 1/04; A61N 5/06

(52) U.S. Cl. CPC A61F 9/04; A61N 1/04; A61N 5/06; A61N 1/32

(57) ABSTRACT

A skin care device according to an embodiment of the present disclosure includes a substrate; a case for covering surface of the substrate; and at least one galvanic current application device electrically connected to the substrate, in which each of the at least one galvanic current application device includes a contact head that forms a contact surface with respect to a user's skin and is electrically connected to the substrate; and a holder provided between the substrate or the case and the contact head, and in which the holder includes an elastic fixing body elastically fixing the contact surface of the contact head to a position spaced apart from the case by a predetermined length.

### TECHNICAL FIELD

The present disclosure relates to a skin care device.

### BACKGROUND ART

The skin can be damaged due to cell aging, repetition of certain facial expressions, continuous exposure to external environments (UV rays, fine dust, or the like), stress, or the like. For example, aging of cells or repetition of specific facial expressions can cause wrinkles on the skin, and continuous exposure to external environments or stress can cause various troubles such as acne and freckles.

Skin care for preventing or minimizing such damage to the skin aims to maintain clean, soft skin without blemishes, and in particular, the most attention is formed on skin care of the face among body parts. Therefore, people want to keep their skin clean by receiving a massage, applying a functional cosmetic product, or using various cleaning products, for facial skin care.

In particular, recently, a device (for example, a mask-type skin care device, or the like) which is attached to or worn on a user's face and outputs light for skin care or applies a microcurrent or the like, has appeared.

### DISCLOSURE

### Technical Problem

An object to be solved by the present disclosure is to provide a skin care device having a galvanic current application device that applies a microcurrent by being in contact with the skin when worn by a user.

Another object to be solved by the present disclosure is to provide a skin care device having a galvanic current application device that can be flexibly in contact with the skin of users having various face shapes.

Another object to be solved by the present disclosure is to provide a skin care device having a galvanic current application device that prevents moisture from penetrating into the device.

### Technical Solution

A skin care device according to an embodiment of the present disclosure includes at least one galvanic current application device configured to be electrically connected to the substrate and to apply a microcurrent when being in contact with the skin of the user.

Each of the at least one galvanic current application device includes a contact head that forms a contact surface with respect to the user's skin and is electrically connected to the substrate; and a holder provided between the substrate or inner case and the contact head, and in which the holder includes an elastic fixing body configured to elastically fix the contact surface of the contact head to a position spaced apart from the inner case by a predetermined length.

The elastic fixed body may be elastically deformed in at least one of a horizontal direction and a vertical direction based on an external force and the separation position of the contact head may be changed by the deformation of the elastic fixed body.

The elastic fixed body may include a fixed body coupled to the contact head; and an elastic body connected to the fixed body.

In the elastic body, a width of a first portion adjacent to the inner case may be longer than a width of a second portion adjacent to the fixed body.

A width of the contact surface may be shorter than the width of the first portion.

According to an embodiment, the width of the contact surface may be shorter than the width of the second portion.

An electric wire for electrical connection between the substrate and the contact head may be connected between the substrate and the contact head through an opening formed in the elastic body and an opening of the inner case.

The opening of the inner case may be blocked by a fastening portion of the holder, and an upper cover and a lower cover respectively fastened to upper and lower parts of the fastening portion.

According to an embodiment, the skin care device may include a plurality of galvanic current application devices, a portion of the plurality of galvanic current application devices may form a positive electrode, and rests may form a negative electrode.

### Advantageous Effect

According to an embodiment of the present disclosure, the galvanic current application device includes a holder that is elastically deformed based on the distance between the skin care device and the user's skin, so that the contact head can be easily in contact with the user's skin having various face shapes.

In addition, since the holder of the galvanic current application device is made of a material having elasticity, such as silicone, it is possible to effectively prevent moisture, dust, or the like from penetrating into the skin care device as well as elastic deformation.

In addition, since the galvanic current application device is implemented in a form that is reduced in width as it goes away from the inner case, it is possible to minimize the blocking of light emitted from the light source provided on the substrate from being irradiated to the skin by the galvanic current application device.

### DESCRIPTION OF DRAWINGS

FIG. 1 is a perspective view illustrating a package including a skin care device and a cradle according to an embodiment of the present disclosure.

FIG. 2 is an exploded perspective view illustrating a skin care device according to an embodiment of the present disclosure.

FIG. 3 is a rear view illustrating a skin care device according to an embodiment of the present disclosure.

FIG. 4 is an exemplary view for explaining the operation of the galvanic current application device illustrated in FIG. 3.

FIG. 5 is an exploded perspective view illustrating the galvanic current application device illustrated in FIG. 3.

FIG. 6 is a view illustrating an example of a plan view and a cross-sectional view of a galvanic current application device.

FIG. 7 is a view illustrating another example of a plan view and a cross-sectional view of a galvanic current application device.

FIGS. 8 to 11 are cross-sectional views for explaining the structure of a galvanic current application device according to embodiments of the present disclosure.

### BEST MODE

Hereinafter, the embodiments disclosed herein will be described in detail with reference to the accompanying drawings, and the same or similar elements are designated with the same reference numbers regardless of the drawing numbers in the drawings and their redundant description will be omitted. The suffixes “module” and “portion” for components used in the following description are given or mixed in consideration of the ease of writing the specification, and thus they do not have distinct meaning or roles by themselves. In addition, when it is determined that the detailed description of the related known technology may obscure the subject matter of embodiments disclosed herein in describing the embodiments, a detailed description thereof will be omitted. Further, the accompanying drawings are intended to facilitate understanding of the embodiments disclosed herein, and the technical spirit disclosed herein is not limited by the accompanying drawings. Therefore, the present disclosure should be construed as including all the changes, equivalents to substitutions included in the spirit and the technical scope of the present disclosure.

Terms including an ordinal number, such as first, second, or the like, may be used to describe various components, but the components are not limited by the terms. The above terms are used only for the purpose of distinguishing one component from another.

When a component is referred to as being “connected” or “accessed” to another component, it may be directly connected or accessed to the other component, but it is understood that other components may exist in between. On the other hand, when it is mentioned that a certain component is “directly connected” or “directly accessed” to another component, it should be understood that no other component exists in between.

The singular expression includes the plural expression unless the context clearly dictates otherwise.

In the present application, terms such as “comprises” or “have” are intended to designate that the features, numbers, steps, operations, components, parts, or combinations thereof described in the specification exist, but it is to be understood that this does not preclude the possibility of addition or existence of one or more other features, numbers, steps, operations, components, parts, or combinations thereof.

Hereinafter, the present embodiment will be described in detail with reference to the accompanying drawings in the present specification.

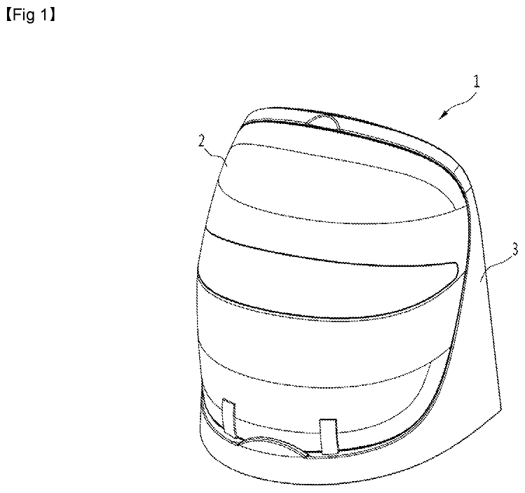

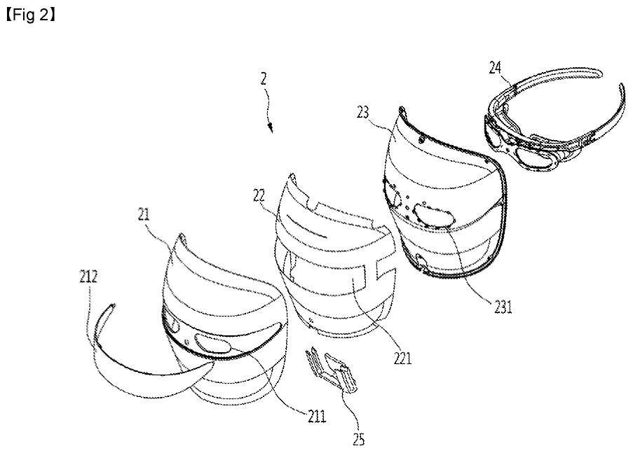

FIG. 1 is a perspective view illustrating a package including a skin care device and a cradle according to an embodiment of the present disclosure, and FIG. 2 is an exploded perspective view illustrating a skin care device according to an embodiment of the present disclosure.

Referring to FIG. 1, the skin care device 2 may be worn on a part of the user's body and output light to the user's skin. In addition, the skin care device 2 may provide various functions for skin care, such as applying a microcurrent by being in contact with the user's skin. Accordingly, the skin care device 2 may promote the activation of the user's skin cells to improve wrinkles, elasticity, and skin tone, or perform skin care operations such as improving skin troubles, removing waste material, and supplying nutrients. The skin care device 2 may constitute one skin care package 1 together with the cradle 3 mounted when the skin care device 2 is charged or stored. As the skin care device 2 is mounted on the cradle 3, a battery (not illustrated) provided in the skin care device 2 may be charged by power supplied from the cradle 3.

The skin care device 2 according to an embodiment of the present disclosure may correspond to a mask type device that is worn on the user's face and outputs light or applies a microcurrent to the user's facial skin. The skin care device 2 may be formed to be generally round according to the shape of a person's face.

The cradle 3 is connected to the skin care device 2 and can be supply power for charging with a battery (not illustrated) provided in the skin care device 2 or a user manipulation device (not illustrated) connected to the skin care device 2. To this end, the cradle 3 may be connected to an external power supply to receive power from the outside.

The cradle 3 may form a seating part for the skin care device 2 to be seated thereon. As illustrated in FIG. 1, the cradle 3 may have a seating part formed to correspond to the outer edge of the skin care device 2, but this is not necessarily the case.

Referring to FIG. 2, the skin care device 2 may include an outer case 21, a substrate 22, an inner case 23, and a wearing device 24. When the user wears the skin care device 2, the outer case 21 may face the outside, and the inner case 23 may face the user's face.

The outer case 21 forms one surface (hereinafter, defined as the front surface) of the skin care device 2 and may protect the substrate 22 provided between the inner case 23 and the outer case 21 from the external shock, contact, or the like. To this end, the outer case 21 may be implemented with a material such as plastic or ceramic.

An opening 211 may be formed in the outer case 21 to secure the user's view when worn by the user. When the user wears the skin care device 2, the user's eyes are positioned in the opening 211, so that the user can secure a view through the opening 211. According to an embodiment, an opening protective cover 212 covering the opening 211 may be provided in order to prevent foreign substances from coming into contact with or colliding with the user's eyes through the opening 211. The opening protective cover 212 may be implemented with a transparent material such as acrylic or plastic.

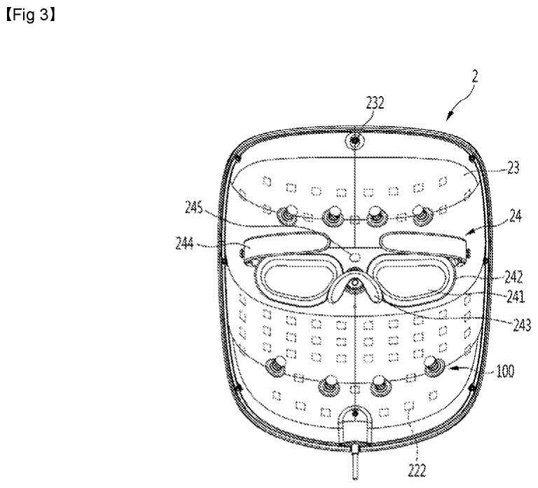

The substrate 22 may include at least one light source 222 (refer to FIG. 3) irradiating light for user's skin care. According to an embodiment, in order to output light of different colors according to the operation mode of the skin care device 2, the at least one light source 222 may include a plurality of LEDs for outputting light of different colors.

For example, the plurality of LEDs may include at least one red LED and at least one blue LED. A red LED may emit red light with a wavelength of about 645 nm to 670 nm. Red light promotes the activation of skin cells, so it can be effective in improving wrinkles, elasticity, and skin tone. A blue LED may emit blue light with a wavelength of about 400 nm to 430 nm. Blue light can be effective in removing various troubles on the skin.

Assuming that the operation mode of the skin care device 2 is divided into an activation promotion mode and a trouble removal mode, when the operation mode is set to the activation promotion mode, the red LED is activated to emit red light to the skin. On the other hand, when the operation mode is set to the trouble removal mode, the blue LED may be activated to emit blue light to the skin. Control of the light source 222 according to the operation mode may be performed by the controller of the skin care device 2 to be described later. According to an embodiment, the plurality of LEDs may further include at least one yellow LED. A yellow LED can emit yellow light with a wavelength of about 580 nm to 600 nm, and the yellow light can be effective in brightening skin tones.

The substrate 22 may include a controller for controlling the light output operation of the at least one light source 222. The controller may be implemented as an integrated circuit (IC), a microcomputer, an embedded processor, an application processor (AP), or the like. According to an embodiment, the controller may be included in a user manipulation device (not illustrated) connected to the skin care device 2.

In addition, a circuit for applying a voltage to the galvanic current application device 100 (refer to FIG. 3) to be described later may be formed on the substrate 22. As a voltage is applied to the galvanic current application device 100, a microcurrent may be applied to the skin of the user in contact with the galvanic current application device 100.

Similar to the outer case 21, an opening 221 may be formed in the substrate 22 to secure a view when worn by a user. As described above, when the user wears the skin care device 2, the user's eyes are positioned in the opening 221, so that the user can secure a view through the opening 221.

Meanwhile, at least a portion of the substrate 22 provided in the skin care device 2 having a mask shape may be formed to have a curved surface. To this end, the substrate 22 may be implemented with a plastic material or the like.

The inner case 23 may form a rear surface of the skin care device 2. The inner case 23 may be formed to be fastened to the outer case 21 and the substrate 22 to cover one surface of the substrate 22. The outer case 21 and the inner case 23 prevent water or other foreign substances from penetrating into the substrate 22 disposed therein, so that the light source 222 or other components disposed on the substrate 22 can be prevented from being failed or damaged.

In addition, when the user wears the skin care device 2, the inner case 23 may be positioned between the substrate 22 and the user's skin to prevent the user's skin from coming into contact with the substrate 22.

The inner case 23 may be implemented with a transparent material such as plastic or acrylic so that light emitted from the light source 222 provided on the substrate 22 is irradiated to the user's facial skin. In particular, the inner case 23 may have a structure so that the light emitted from the light source 222 is evenly irradiated to the skin or the light is concentrated to a specific part.

The inner case 23 may also include an opening 231 for securing a user's view, like the outer case 21 and the substrate 22.

Meanwhile, when worn by the user, a plurality of galvanic current application devices 100 that are in contact with the skin may be formed on the rear of the inner case 23. The plurality of galvanic current application devices 100 may be electrically connected to the substrate 22 and implemented to apply a microcurrent to the user's skin. Specific embodiments related to the galvanic current application device 100 will be described later with reference to FIGS. 3 to 11.

The wearing device 24 may fix the skin care device 2 to the user as the user wears the skin care device 2. The wearing device 24 may be fastened to the inner case 23. For example, the wearing device 24 may have a shape of glasses that can be worn while being seated on the user's nose and ears.

In particular, the wearing device 24 according to an embodiment of the present disclosure may include a wear detection sensor for effectively detecting whether the user is wearing the wearing device 24. A more specific structure and configuration of the wearing device 24 will be described later with reference to FIG. 3.

According to an embodiment, a chin fixing device 25 may be provided under the skin care device 2. The chin fixing device 25 may be detachably coupled to at least one of the outer case 21 and the inner case 23. When the user wears the skin care device 2, the chin fixing device 25 may be in contact with the user's chin to stably fix the skin care device 2 to the user's face.

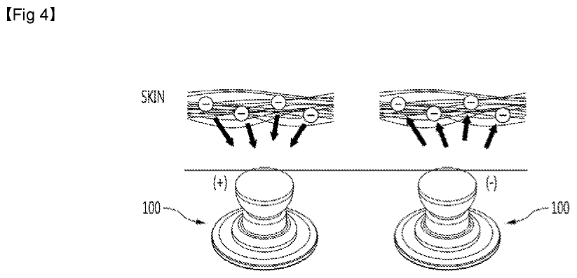

FIG. 3 is a rear view illustrating a skin care device according to an embodiment of the present disclosure, and FIG. 4 is an exemplary view for explaining the operation of the galvanic current application device illustrated in FIG. 3.

Referring to FIG. 3, the wearing device 24 may include an opening 241, a face contact part 242, a nose seat part 243, a wearing fixing part 244, and a wearing detection sensor 245.

Like the outer case 21, the substrate 22, and the inner case 23, the opening 241 may be formed to secure a view when worn by a user. The opening 241 may include a first opening and a second opening formed at positions corresponding to the left and right eyes of the user, respectively. According to an embodiment, when the opening protective cover 212 is not provided in the outer case 21, lens may be provided in the opening 241 of the wearing device 24. By the opening protective cover 212 or the lens, it is possible to prevent a foreign substance from coming into contact with or collide with the user's eyes.

Comparing the openings formed in each of the outer case 21, the substrate 22, the inner case 23, and the wearing device 24, the size of the opening 221 formed in the substrate 22 may be greater than or equal to the size of the remaining openings 211, 231, 241. In other words, the boundary surface of the opening 221 formed in the substrate 22 is spaced apart from the user's left and right eyes by a predetermined distance, thereby preventing the light source 222 of the substrate 22 from being disposed to be close to the user's left and right eyes. Meanwhile, the size of the opening 241 formed in the wearing device 24 is formed to be smaller than or equal to the size of the remaining openings 211, 221, and 231 to effectively secure the user's view.

The face contact part 242 may be formed along the boundary surface of the opening 241. When the user wears the skin care device 2, the face contact part 242 may be composed of a first face contact part and a second face contact part which are respectively in contact with the skin surface located around the user's left eye and the skin surface located around the right eye of the user. As the face contact part 242 is in contact with the skin surface located around both eyes of the user, it is possible to prevent the light emitted from the light source 222 of the substrate 22 from being irradiated to the user's eyes. The face contact part 242 may be made of a material such as rubber or silicone and may have an opaque color to minimize light transmission.

The nose seat part 243 may be disposed between the first opening and the second opening or between the first face contact part and the second face contact part and may be seated on the user's nose when worn by the user. In order to be seated on the user's nose, the nose seat part 243 may be formed to be round to correspond to the shape of a person's nose.

The wearing fixing part 244 may be fixed to the user's body by being seated on the user's ear or in close contact with the user's head circumference when worn. By the nose seating part 243 and the wearing fixing part 244, the skin care device 2 may be worn and fixed on the user's body.

The wear detection sensor 245 may detect whether the skin care device 2 is worn on the user's body. For example, the wear detection sensor 245 may be implemented as a proximity sensor. When the wear detection sensor 245 is implemented as a proximity sensor, the proximity sensor may detect that a part of the user's body (for example, face) approaches the proximity sensor as the skin care device 2 is worn. The proximity sensor may transmit a detection signal according to detection of proximity of a user's body part to a controller (not illustrated; processor, or the like) of the skin care device 2. The controller of the skin care device 2 may detect whether the skin care device 2 is worn based on a detection signal transmitted from the proximity sensor.

According to an embodiment, when the user wears the skin care device 2, in order to prevent a false detection due to proximity of other parts such as the user's hand, when the controller of the skin care device 2 continuously receives the detection signal for a predetermined time or longer, it may be detected that the skin care device 2 is normally worn.

The wear detection sensor 245 may be electrically connected to the substrate 22.

In particular, as illustrated in FIG. 3, the wear detection sensor 245 may be disposed between the first opening and the second opening, or between the first face contact part and the second face contact part, and above the nose seat part 243. In other words, the wear detection sensor 245 may be positioned to correspond to the forehead or the middle of the forehead when worn by the user and may detect the proximity of the user's forehead or the middle of the forehead.

A person's face shape may be different from each other, and the distance between the skin care device 2 and each part of the face may also be partially different according to the face shape. However, in general, when the skin care device 2 is worn, the distance between the skin care device 2 and the middle of the forehead may have a relatively small difference depending on the face shape. Accordingly, in the skin care device 2 according to an embodiment of the present disclosure, the wear detection sensor 245 is disposed at a position corresponding to the user's middle of the forehead, thereby more effectively detecting whether the user is wearing the skin care device 2 regardless of the user's face shape.

Continuing to refer to FIG. 3, the skin care device 2 according to an embodiment of the present disclosure may further include a plurality of galvanic current application devices 100.

The plurality of galvanic current application devices 100 may protrude toward the rear of the inner case 23 by a predetermined length and may be in contact with the user's skin when worn by the user.

The plurality of galvanic current application devices 100 may be disposed to be spaced apart from each other. In addition, each of the plurality of galvanic current application devices 100 may be disposed at a position corresponding to a portion of the user's face requiring intensive skin care, such as a forehead and cheeks. Each of the plurality of galvanic current application devices 100 may be fastened to the inner case 23, but is not limited thereto.

Meanwhile, the plurality of galvanic current application devices 100 may be electrically connected to the substrate 22 to apply a microcurrent to the user's skin. To this end, some of the plurality of galvanic current application devices 100 may form an a positive electrode (+), and others may form a negative electrode (−). According to an embodiment, the polarity of each of the galvanic current application devices 100 may be periodically alternated.

Referring to FIG. 4, when a plurality of galvanic current application devices 100 are in contact with the user's skin, the positive electrode (+) and negative electrode (−) of the microcurrent (galvanic current) applied to the skin through the galvanic current application device 100 have different effects, and when molecules are ionized and electrified, by using the property that the same polarity is repelled and the opposite polarity is attracted, providing the function of penetrating active ingredients or discharging waste material out of the skin.

As illustrated in the left side of FIG. 4, when the waste material has a negative polarity (−), the waste material may be discharged out of the skin as it moves to the galvanic current application device 100 forming the positive electrode (+).

In addition, when the active ingredient forms the negative electrode (−) as illustrated on the right side of FIG. 4, the active ingredient can effectively penetrate into the skin by the galvanic current application device 100 forming the negative electrode (−).

Although not illustrated, according to an embodiment, the skin care device 2 may have other additional components in contact with the user's skin. In this case, the plurality of galvanic current application devices 100 may all form the same polarity, and the additional configuration may form a polarity different from that of the galvanic current application device 100.

Meanwhile, since the shape of the skin care device 2 is fixed and the user's face shape varies, the distance between the skin care device 2 and the user's skin may vary according to the face shape of the user wearing the skin care device 2.

For example, when the distance between the skin care device 2 and the user's skin exceeds a predetermined distance, the skin care function may not be provided because the galvanic current application device 100 does not be in contact with the skin.

Alternatively, when the distance between the skin care device 2 and the user's skin is too close, the galvanic current application device 100 presses the skin, so that the user may feel uncomfortable.

According to the present embodiment, a galvanic current application device having a structure that can be flexibly in contact with skin for users having various face shapes may be provided.

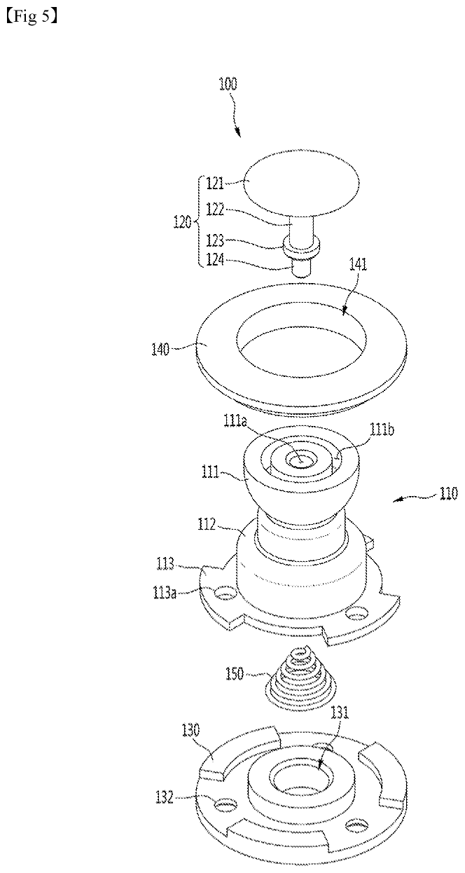

Hereinafter, various embodiments related to the structure of the galvanic current application device will be described with reference to FIGS. 5 to 11.

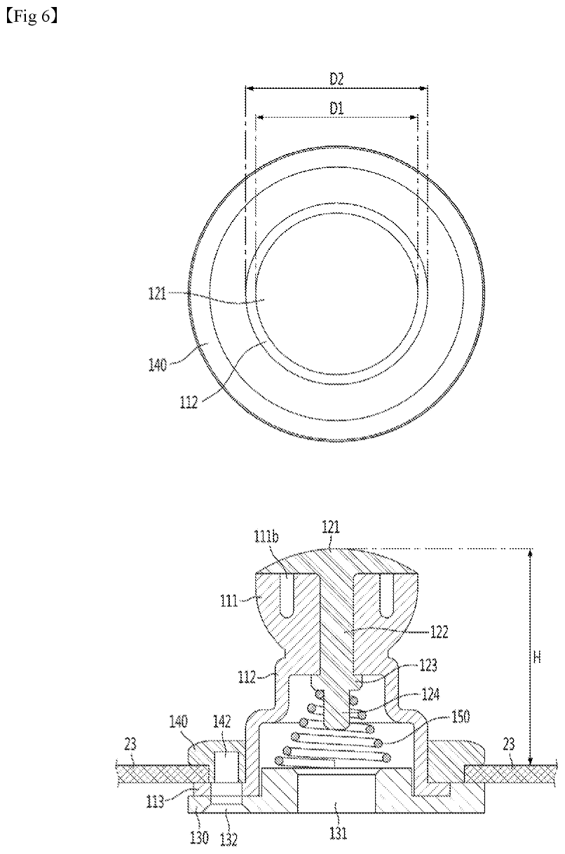

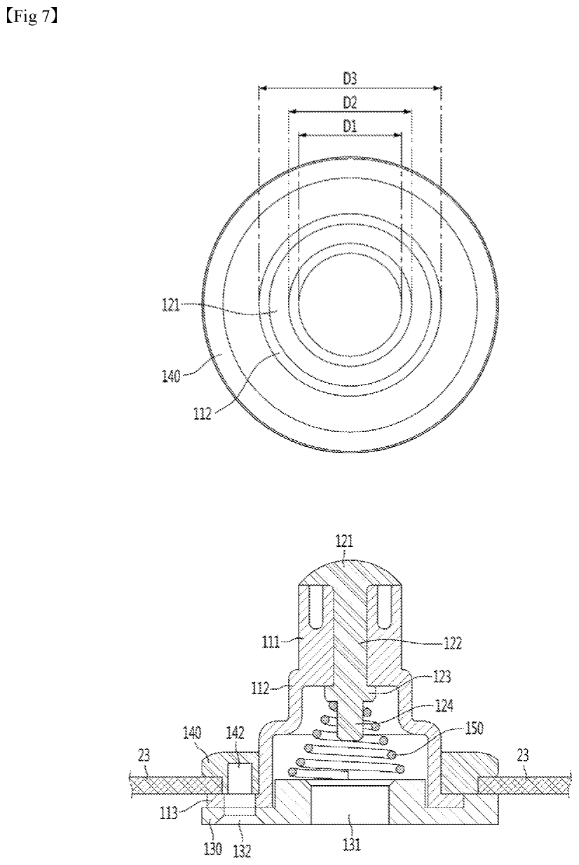

FIG. 5 is an exploded perspective view illustrating the galvanic current application device illustrated in FIG. 3, FIG. 6 is a view illustrating an example of a plan view and a cross-sectional view of a galvanic current application device, and FIG. 7 is a view illustrating another example of a plan view and a cross-sectional view of a galvanic current application device.

For convenience of description below, the contact plate 121 of the contact head 120 is defined as corresponding the upper side of the galvanic current application device 100, and the lower cover 130 is defined as corresponding to the lower side of the galvanic current application device 100.

Referring to FIGS. 5 to 7, the galvanic current application device 100 may include a holder 110 and a contact head 120.

FIG. 5 illustrates that the holder 110 has a jar shape or a cylindrical shape and the contact plate 121 of the contact head 120 has a circular plate shape, but the shapes of the holder 110 and the contact plate 121 are not limited thereto.

The holder 110 may be formed between the substrate 22 or the inner case 23 and the contact head 120 to support the contact head 120 with respect to the substrate 22 or the inner case 23.

Specifically, the holder 110 may include an elastic fixed body including a fixed body 111 and an elastic body 112.

The fixed body 111 may be coupled (eg, fitted) to the contact head 120 to support the contact head 120 with respect to the substrate 22 or the inner case 23.

For example, the fixed body 111 may include a first opening 111a formed in a vertical direction. The fixing bar 122, the separation prevention portion 123, and the terminal 124 of the contact head 120 may be inserted through the first opening 111a and fixed to the holder 110.

The upper surface of the fixed body 111 is formed to correspond to the lower surface of the contact plate 121, so that a gap between the fixed body 111 and the contact plate 121 can be minimized. Accordingly, it is possible to prevent damage or failure of components provided in the substrate 22 by preventing the liquid such as moisture from penetrating into the skin care device 2 through the gap.

According to an embodiment, a waterproof groove 111b may be formed on the upper surface of the fixed body 111. The waterproof groove 111b may be formed in a ring shape at a predetermined position between the outer circumferential surface of the fixed body 111 and the first opening 111a. When the liquid flows therein through the gap between the fixed body 111 and the contact plate 121, the inflow liquid may be accommodated in the waterproof groove 111b and thus may not penetrate into the skin care device 2.

The elastic body 112 may be formed to extend below the fixed body 111. The elastic body 112 may be connected to the fastening portion 113 forming the lower part of the holder 110.

The elastic body 112 may be formed to have a lower width wider than an upper width. For example, the elastic body 112 may form a multi-stage structure or a bellows-type structure, thereby enabling smoother elastic deformation in each of the horizontal and vertical directions, and preventing damage during elastic deformation. Although the elastic body 112 having a two-stage structure is illustrated in FIG. 5, the shape of the elastic body 112 may be similarly changed within a range apparent to those skilled in the art. In addition, since the upper width of the elastic body 112 is narrowed, it is possible to minimize blocking of the light irradiated toward the skin from the light source 222 provided on the substrate 22 by the elastic body 112.

Meanwhile, a second opening may be formed in a lower part of the elastic body 112, and a space may be formed in the elastic body 112. In this case, the first opening 111a and the inner space of the elastic body 112 may communicate with each other.

A configuration such as an electric wire (not illustrated) for applying a voltage to the contact head 120 may electrically connect the substrate 22 and the contact head 120 through the second opening and the inner space.

The elastic body 112 may be implemented with a material having elasticity. For example, the elastic body 112 may be implemented with silicon. In addition, the holder 110 may be integrally formed, wherein the fixed body 111, the elastic body 112, and the fastening portion 113 may all be implemented with silicon. According to an embodiment, the elastic body 112 may be made of a transparent or translucent elastic material to minimize blocking of light irradiated from the light source 222 of the substrate 22.

The fastening portion 113 may be formed under the elastic body 112. The fastening portion 113 may be fastened to the substrate 22 or the inner case 23 to fix the galvanic current application device 100 to the skin care device 2.

For example, the fastening portion 113 may have a shape extending by a predetermined length in the horizontal direction from the lower part of the elastic body 112.

Meanwhile, the galvanic current application device 100 may further include at least one cover for fixing the galvanic current application device 100 to the skin care device 2 by being fastened to the fastening portion 113.

For example, the at least one cover may include a lower cover 130 fastened to a lower part of the fastening portion 113 and an upper cover 140 fastened to an upper part of the fastening portion 113.

At least one fastening hole 113a is formed in the fastening portion 113, and at least one lower fastening hole 132 formed in a position corresponding to the at least one fastening hole 113a may be formed in the lower cover 130. In addition, at least one fastening groove 142 formed at a position corresponding to the at least one fastening hole 113a may be formed in the upper cover 140.

A fastening member such as a screw (not illustrated) penetrates the lower fastening hole 132 and the fastening hole 113a and is inserted into the fastening groove 142, thereby fastening the fastening portion 113 to a portion between the lower cover 130 and the upper cover 140.

For example, the lower cover 130 may be fastened to be in contact with a bottom surface of the fastening portion 113 and a portion of an inner circumferential surface of the elastic body 112. In addition, the upper cover 140 may be fastened to be in contact with the upper surface of the fastening portion 113 and a portion of the outer circumferential surface of the elastic body 112. Accordingly, since the upper cover 140 and the holder 110 are strongly attached to each other, it is possible to prevent moisture or the like from penetrating between the upper cover 140 and the holder 110.

The lower cover 130 may have an opening 131 through which the above-described configuration, such as an electric wire, passes. An opening 141 through which the elastic fixing bodies 111 and 112 pass may be formed in the upper cover 140. A portion of the upper cover 140 may be seated on one surface of the inner case 23 when fastened.

Meanwhile, an opening corresponding to each of the galvanic current application device 100 may be formed in the inner case 23. As illustrated in FIGS. 6 and 7, the fastening portion 113 is located below the inner case 23, the fixed body 111 is located above the inner case 23, and the elastic body 112 may be connected a portion between the fixed body 111 and the fastening portion 113 through the opening.

In addition, the lower cover 130 may be disposed below the opening of the inner case 23, and at least a portion of the upper cover 140 may be disposed above the opening of the inner case 23. In other words, since the opening of the inner case 23 is blocked by the lower cover 130, the holder 110, and the upper cover 140, it is possible to prevent moisture or the like from penetrating into the substrate 22 through the opening.

According to an embodiment, an elastic member (for example, spring 150) for elastic deformation of the holder 110 may be further provided inside the elastic body 112. The elastic member 150 may be installed between the upper surface of the lower body 130 and the lower part of the separation prevention portion 123, but is not limited thereto.

The contact head 120 is electrically connected to the substrate 22 and may apply a microcurrent upon being in contact with the user's skin. For example, a component (circuit or the like) for supplying a galvanic current is formed on the substrate 22, and the contact head 120 may be electrically connected to the component through an electric wire or a conductive elastic gasket. The contact head 120 may be implemented with a conductive metal or the like.

For example, the contact head 120 may include a contact plate 121 that forms a contact surface with the skin. The upper surface of the contact plate 121 may form the contact surface, and the lower surface of the contact plate 121 may contact the upper surface of the fixed body 111.

In some embodiments, the contact surface may be convex for uniform contact with the skin.

The contact head 120 may further include a fixing bar 122 extending downwardly of the contact plate 121, a separation prevention portion 123 formed below the fixing bar 122, and a terminal 124 extending downward of the separation prevention portion 123.

The fixing bar 122, the separation prevention portion 123, and the terminal 124 may be inserted and fixed into the inner space of the elastic fixing body.

The fixing bar 122 may be accommodated and fixed in a space corresponding to the first opening 111a of the internal space, and the separation prevention portion 123 and the terminal 124 may be accommodated in the internal space of the elastic body 112.

The width (or thickness) of the separation prevention portion 123 may be longer than the width (or thickness) of the fixing bar 122. In addition, the width of the separation prevention portion 123 may be longer than the width of the first opening 111a. In other words, the separation prevention portion 123 may prevent the contact head 120 from being separated through the upper part of the holder 110.

The terminal 124 may extend to the lower side of the separation prevention portion 123 by a predetermined length and may be connected to an electric wire (not illustrated) connected to the substrate 22. According to an embodiment, when the substrate 22 is provided with a configuration such as a conductive elastic gasket (not illustrated) instead of an electric wire, the terminal 124 may be formed to extend through an opening 131 of the lower cover 130 to contact the conductive elastic gasket.

According to an embodiment, the contact head 120 may not include the terminal 124, and in this case, a configuration for supplying electric current such as an electric wire may be connected to the fixing bar 122 or the separation prevention portion 123.

Referring to FIGS. 6 to 7, when the user wears the skin care device 2, a separation by a predetermined distance may occur between the inner case 23 and the user's skin by the wearing device 24 or the like. In addition, the separation distance between the inner case 23 and the skin may vary according to the shape of the user's face.

Based on this, the upper surface, ie, the contact surface, of the contact plate 121 of the contact head 120 may be formed to have a predetermined length (or height H) from the inner case 23. In addition, the holder 110 is elastically deformed when the distance between the inner case 23 and the user's skin is less than a predetermined distance, so that when the contact surface and the skin are in contact with each other, it is possible to prevent a pressure of a predetermined size or more from being applied to the user's skin.

For example, the height H between the contact surface and the inner case 23 may be 1.5 cm to 2.5 cm, but is not limited thereto.

Meanwhile, as described above, at least one light source 222 (LED or the like) may be provided on the substrate 22, and the light source 222 can irradiate light to the user's skin when the skin care device 2 is used.

At this time, since the galvanic current application device 100 is formed to have a predetermined height (H) from the inner case 23, the light irradiated from the light source 222 is blocked by the galvanic current application device 100, so that the light may not be irradiated smoothly to the skin of the user.

Accordingly, the width D1 of the contact plate 121 and the fixed body 111 is shorter than the lower width D2 of the elastic body 112, and blocking of the light irradiated from the light source 222 disposed around the galvanic current application device 100 can be minimized.

According to an embodiment, as illustrated in FIG. 7, the width D1 of the contact plate 121 and the fixed body 111 is shorter than the lower width D2 and the upper width D3 of the elastic body 112. Although not illustrated, the width of the contact plate 121 and the fixed body 111 is formed as a value between the lower width D2 of the elastic body 112 and the width of the upper body 140, and it may be also possible to increase the contact area with the skin of the user.

In other words, the galvanic current application device 100 according to an embodiment of the present disclosure includes a holder 110 that is elastically deformed based on the distance between the skin care device 2 (for example, the inner case 23) and the user's skin, and thus the contact head 120 is easily in contact with the user's skin having various face shapes.

In addition, since the holder 110 of the galvanic current application device 100 is made of a material having elasticity such as silicone, it is possible to effectively prevent not only the above-described elastic deformation but also the penetration of moisture into the skin care device 2.

In addition, the galvanic current application device 100 is implemented in a form in which the width decreases as it goes away from the inner case 23, thereby minimizing the blocking of light irradiated from the light source 222 provided on the substrate 22.

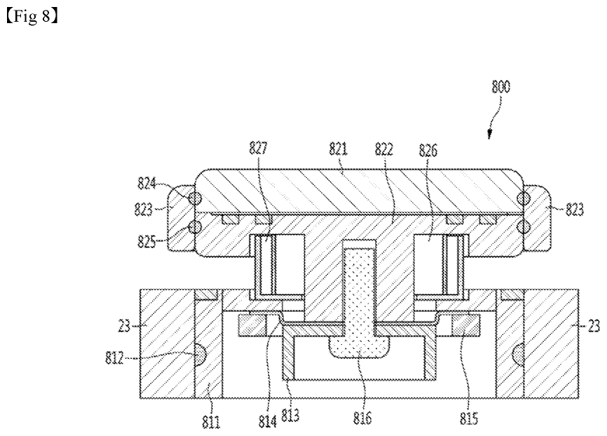

Hereinafter, various embodiments related to the structure of the galvanic current application device will be described with reference to FIGS. 8 to 11.

FIGS. 8 to 11 are cross-sectional views for explaining the structure of a galvanic current application device according to embodiments of the present disclosure.

Referring to FIG. 8, the galvanic current application device 800 may be implemented in the form of a module mounted on the opening of the inner case 23.

The galvanic current application device 800 may include a first body part mounted to the opening of the inner case 23 and a second body part fastened to an upper part of the first body part.

The first body part may include an outer body 811 in contact with the opening of the inner case 23. A first sealing member 812 for waterproofing and/or dustproofing may be formed between the outer body 811 and the opening of the inner case 23. The first sealing member 812 may be implemented as a silicon ring, but is not limited thereto.

The first body part may further include an inner body 813 formed inside the outer body 811. A fastening hole for fastening the first body part and the second body part may be formed in the inner body 813.

The second body part forms a contact surface with the skin and may include a contact electrode 821 for applying a microcurrent to the skin, a support body 822 supporting the contact electrode 821, and a side fixing body 823 surrounding the contact electrode 821 and a portion of the side of the support body 822.

The contact electrode 821 may correspond to the contact plate 121 of the contact head 120 described above.

The support body 822 may include a support plate supporting the contact electrode 821 and a fastening portion extending downward of the support plate. A fastening groove for fastening with the inner body 813 may be formed in the fastening portion. By inserting a fastening member (a screw or the like) through the fastening hole and the fastening groove of the inner body 813, the first body part and the second body part may be fastened to each other.

Meanwhile, the second body part may further include a second sealing member 824 for sealing between the contact electrode 821 and the side fixing body 823, and a third sealing member 825 for sealing between the supporting body 822 and the side fixing body 823. According to an embodiment, the support body 822 and the side fixing body 823 may be integrally formed.

With continued reference to FIG. 8, the first body part may further include a fourth sealing member 814 for waterproofing and dustproofing between the outer body 812, the inner body 813, and the support body 822. The fourth sealing member 814 may be formed between the outer body 812 and the inner body 813 and between the inner body 813 and the support body 822.

A fixing ring 815 may be provided in a space between the outer body 812 and the inner body 813. The fixing ring 815 is formed to be in close contact with the fourth sealing member 814 located between the outer body 812 and the inner body 813 to one surface of the outer body 812, and thus it is possible to effectively prevent moisture or the like from penetrating into the space between the the outer body 812 and the inner body 813.

Meanwhile, the outer circumferential surface of the support body 822 and the outer body 811 may be spaced apart from each other by a predetermined distance. Accordingly, the second body part may be moved by a predetermined distance in the horizontal direction with respect to the first body part.

In addition, the second body part may include an elastic member 826 (for example, a spring) surrounding a portion of the outer circumferential surface of the support body 822. The elastic member 826 may be disposed between the lower surface of the support plate of the support body 822 and the upper surface of the outer body 811, thereby elastically fixing the second body part with respect to the first body part in the vertical direction. In other words, when an external force is applied, the second body part may move in a vertical direction and a horizontal direction with respect to the first body part, respectively.

According to an embodiment, the galvanic current application device 800 may further include the support body 822, the elastic member 826, and a fifth sealing member 827 for waterproofing and dustproofing between the elastic member 826 and the outer body 811. In this case, the lower part of the elastic member 826 may be seated on the upper part of the fifth sealing member 827.

In other words, by the first to fifth sealing members 812, 814, 824, 825, and 827, it is possible to effectively prevent external moisture, dust, or the like from flowing into the substrate 22.

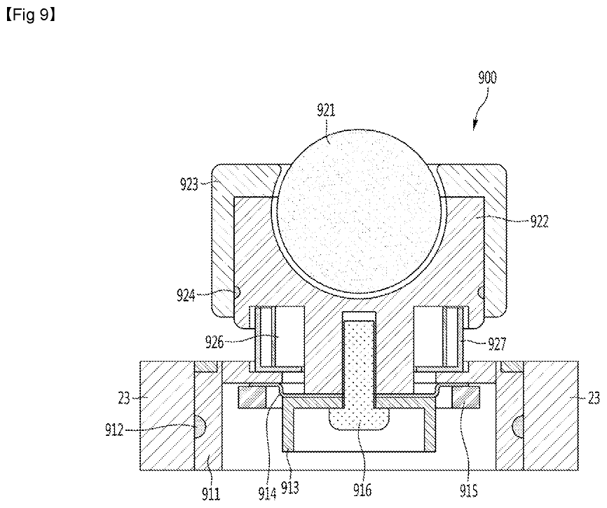

Referring to FIG. 9, a description of a configuration having substantially the same shape and arrangement as that illustrated in the galvanic current application device 800 of FIG. 8 will be omitted.

The second body part of the galvanic current application device 900 may include a spherical contact electrode 921. The second body part may include a support body 922 forming a contact electrode accommodation part thereon, and an outer cover 923 surrounding the side and upper surfaces of the support body 922 and accommodating the contact electrode 921 together with the contact electrode accommodation part 923. The contact electrode 921 accommodated in the contact electrode accommodation part may be prevented from being separated to the outside by the outer cover 923.

The contact electrode 921 may be rotated in the contact electrode accommodation part. Accordingly, even if a user's movement occurs while wearing the skin care device 2, the contact between the contact electrode 921 and the skin may be effectively maintained.

In addition, as described above in FIG. 8, the second body part may move in a horizontal direction and a vertical direction with respect to the first body part.

Meanwhile, a first sealing member 912 may be formed between the inner case 23 and the outer body 911. A second sealing member 914 may be formed between the outer body 911, the inner body 913, and the support body 922. A third sealing member 924 may be formed between the support body 922 and the outer cover 923, and a fourth sealing member 927 formed a portion between the lower surface of the contact electrode accommodation part and the upper surface of the outer body 911 may be provided on the outside of the elastic member 926. The first to fourth sealing members 912, 914, 924, and 927 may effectively prevent external moisture, dust, or the like from flowing into the substrate 22.

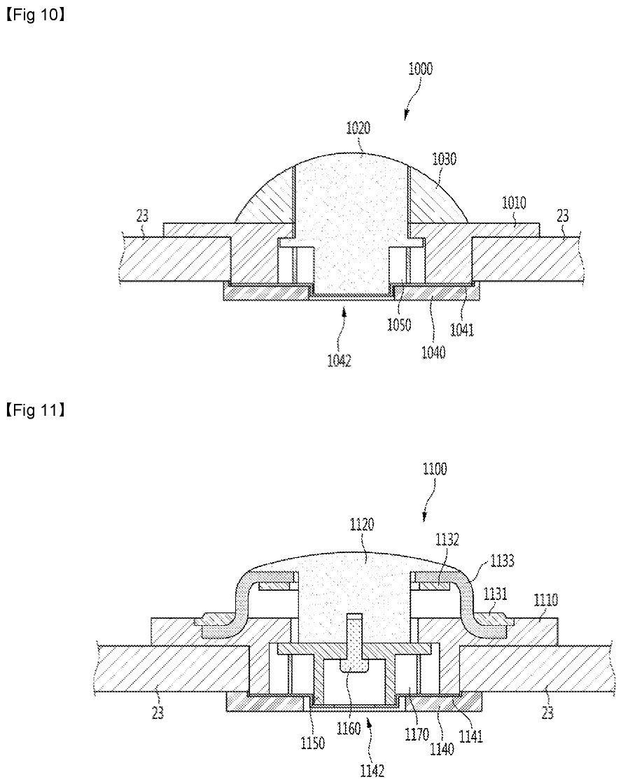

Referring to FIG. 10, the galvanic current application device 1000 may include a body 1010, a contact electrode 1020, an upper cover 1030, and a lower cover 1040.

The body 1010 may generally have a ring shape. In addition, the upper part of the body 1010 may have a larger outer circumferential surface than the lower part thereof and may be seated on the upper surface of the inner case 23.

At least a portion of the contact electrode 1020 may be exposed through the inside of the body 1010 to the upper part of the body 1010. The exposed contact electrode 1020 may come into contact with the user's skin.

An upper cover 1030 may be formed in an area corresponding to the upper part of the body 1010 and the outer circumferential surface of the contact electrode 1020. The upper cover 1030 is implemented with a material such as silicone and may be appropriately elastically deformed according to the strength of contact with the skin.

The lower cover 1040 may be fastened to a lower part of the body 1010. When the lower cover 1040 is fastened, the inner case 23 is positioned between a part of the lower cover 1040 and a part of the body 1010, so that the galvanic current application device 1000 can be fixed to the inner case 23.

According to an embodiment, a sealing member 104 may be formed between the lower cover 1040 and the body 1010.

Meanwhile, the contact electrode 1020 may move in the vertical direction according to the strength of contact with the user's skin. The contact electrode 1020 may include a protrusion that is accommodated in the inner space of the body 1010 and protrudes in a horizontal direction compared to the rest of the body 1010. The width of the protrusion is formed to be wider than the width of the opening of the body 1010, so that the contact electrode 1020 can be prevented from separating to the outside through the upper part of the body 1010.

According to an embodiment, the contact electrode 1020 may further include a terminal part extending under the protrusion. The width of the terminal part is formed to be equal to or smaller than the width of the opening 1042 of the lower cover 1040, so that the contact electrode 1020 may pass through the opening 1042 of the lower cover 1040 when the contact electrode 1020 moves downward. An electric wire or the like for electrical connection may be provided between the terminal part and the substrate 22.

Meanwhile, an elastic member 1050 is provided between the protrusion and the lower body 1040 to elastically fix the contact electrode 1020.

Referring to FIG. 11, the body 1110 and the lower cover 1140 may be formed to be generally similar to the body 1010 and the lower cover 1040 illustrated in FIG. 10.

Meanwhile, the contact electrode 1120 may include a contact plate that forms a contact surface with the skin, and a fastening portion extending below the contact plate. The width of the fastening portion may be narrower than the width of the contact plate, and thus the bottom surface of the contact plate may be present.

A first cover 1131, a second cover 1132, and a third cover 1133 may be formed between the contact electrode 1120 and the body 1110.

The first cover 1131 may be formed on a portion of the upper surface of the body 1110, and a portion of the third cover 1133 may be fixed between the first cover 1131 and the body 1110.

The second cover 1132 may be formed under the lower surface of the contact plate, and another portion of the third cover 1133 may be fixed between the second cover 1132 and the lower surface of the contact plate.

The third cover 1133 may be implemented with a material such as silicon having softness. A portion of the third cover 1133 is fixed between the first cover 1131 and the body 1110, and the other portion thereof is fixed to a portion between the second cover 1132 and the bottom surface of the contact plate, so that inflow between the contact electrode 1120 and the body 1110 of external moisture or the like may be prevented.

Meanwhile, the width of the fastening portion of the contact electrode 1120 may be smaller than the width of the opening of the body 1110. Accordingly, when an external force is applied, the contact electrode 1120 may move in a horizontal direction and a vertical direction with respect to the body 1110.

A support body 1150 which is fastened to the contact electrode 1120 via a fastening member such as a screw 1160 to support the contact electrode 1120 may be formed under the contact electrode 1120. The support body 1150 may have an overall shape similar to that of the protrusion and the terminal part of FIG. 10.

The support body 1150 may include a support plate supporting the contact electrode 1120 and an extension body extending below the support plate. The width of the extension body is formed to be smaller than the width of the opening 1142 of the lower cover 1140, so that the contact electrode 1120 may pass through the opening 1142 when the contact electrode 1120 moves downward.

An elastic member 1170 (for example, a spring) is provided between the bottom surface of the support plate and the upper surface of the lower cover 1140 to elastically fix the contact electrode 1120.

The above description is merely illustrative of the technical spirit of the present disclosure, and various modifications and variations will be possible without departing from the essential characteristics of the present disclosure by those skilled in the art to which the present disclosure pertains.

Therefore, the embodiments disclosed in the present disclosure are not intended to limit the technical spirit of the present disclosure, but to explain, and the scope of the technical spirit of the present disclosure is not limited by these embodiments.

The protection scope of the present disclosure should be construed by the following claims, and all technical ideas within the scope equivalent thereto should be construed as being included in the scope of the present disclosure.

1. A skin care device comprising:
a substrate;
a case disposed over a surface of the substrate; and
a galvanic current application device configured to be electrically connected to the substrate,
wherein the galvanic current application device includes:
a contact head forming a contact surface with respect to a user's skin and electrically connected to the substrate to supply current to the user's skin; and
a holder disposed between the substrate and the contact head or disposed between the case and the contact head, and
wherein the holder includes an elastic fixed body configured to elastically fix the contact surface of the contact head to a position spaced apart from the case by a predetermined length.

2. The skin care device of claim 1,
wherein the elastic fixed body is configured to be elastically deformed in at least one of a horizontal direction and a vertical direction based on an external force.

3. The skin care device of claim 1,
wherein the holder further includes a fastening portion configured to fix the galvanic current application device to the substrate or to the case, and
wherein the elastic fixed body includes:
a fixed body coupled to the contact head; and
an elastic body connected between the fixed body and the fastening portion.

4. The skin care device of claim 3, further comprising:
a light source disposed on the surface of the substrate,
wherein the elastic body includes a first portion adjacent to the fastening portion and a second portion connected to the fixed body, and
wherein a width of the first portion of the elastic body is greater than a width of the second portion of the elastic body.

5. The skin care device of claim 4,
wherein a width of the contact surface of the contact head is less than the width of the first portion of the elastic body.

6. The skin care device of claim 5,
wherein the width of the contact surface of the contact head is less than the width of the second portion of the elastic body.

7. The skin care device of claim 4,
wherein the elastic body has a multi-stage structure or a bellows structure.

8. The skin care device of claim 3, wherein the holder further includes:
a first opening formed in the fixed body; and
a second opening connected to the first opening and formed in the elastic body,
wherein the contact head includes:
a contact plate forming the contact surface;
a fixing bar extending from the contact plate away from the contact surface and disposed in the first opening;
a separation prevention portion disposed on the fixing bar and spaced from the contact plate, a width of the separation prevention portion being greater than a width of the fixing bar; and
a terminal extending from the separation prevention portion, and
wherein the separation prevention portion and the terminal are disposed in the second opening.

9. The skin care device of claim 8, further comprising:
an electric wire connected to the substrate and the terminal and extending through the second opening, the electric wire electrically connecting the substrate to the contact head.

10. The skin care device of claim 8, further comprising:
a spring disposed in the second opening and surrounding at least a portion of the terminal.

11. The skin care device of claim 3,
wherein the case includes an opening,
wherein the galvanic current application device is disposed in the opening of the case, and
wherein a portion of the elastic fixed body is disposed in the opening of the case.

12. The skin care device of claim 11,
wherein the galvanic current application device further includes:
a lower cover disposed under the opening of the case and fastened to a lower surface of the fastening portion; and
an upper cover at least partially disposed over the opening of the case and fastened to an upper surface of the fastening portion, and
wherein the opening of the case is blocked by the lower cover, the upper cover, and the fastening portion.

13. The skin care device of claim 1,
wherein the contact head includes metal, and
wherein the holder includes silicone.

14. The skin care device of claim 1,
wherein the galvanic current application device is among a plurality of galvanic current application devices of the skin care device, and
wherein a first group of the plurality of galvanic current application devices form a positive electrode, and a second group form a negative electrode.

15. The skin care device of claim 14,
wherein a polarity of each of the plurality of galvanic current application devices is periodically alternated.

16. A skin care device comprising:
a substrate having a light source disposed on a surface of the substrate;
a case disposed on the surface of the substrate; and
a galvanic current application device electrically connected to the substrate to supply a current to a user's skin, the galvanic current application device including:
a contact head spaced from an upper side of the case by a predetermined distance and electrically connected to the substrate;
a holder including:
a fixed body coupled to the contact head;
a fastening portion positioned below the case; and
an elastic body connected between the fixing part and the fastening portion;
a lower cover fastened to a lower surface of the fastening portion; and
an upper cover fastened an upper surface of the fastening portion.

17. The skin care device of claim 16, wherein the galvanic current application device is among a plurality of galvanic current application devices of the skin care device,
wherein the case includes a plurality of openings, each of the plurality of galvanic current application devices being disposed in a respective one of the plurality of openings of the case, and
wherein each opening of the case is blocked by the lower cover, the upper cover, and the fastening portion of a respective one of the plurality of galvanic current application devices.

18. The skin care device of claim 16, wherein the contact head includes:
a contact plate forming the contact surface;
a fixing bar extending from the contact plate away from the contact surface and disposed in the elastic body; and
a separation prevention portion disposed on a lower side of the fixing bar, a width of the separation prevention portion being greater than a width of the fixing bar.

19. The skin care device of claim 18, wherein the holder further includes a spring having a first end contacting the contact head and a second end contacting the lower cover, and
wherein the spring contacts the separation prevention portion of the contact.

20. The skin care device of claim 16, further comprising a wearing device attached to the case and including:
a wearing fixing part configured to be fixed to the user's body; and
a wearing detection sensor configured to detect if the user is wearing the skin care device.
